(19) United States
(12) Patent Application Publication
(10) Pub. No.: US 2022/0155294 A1
(43) Pub. Date: May 19, 2022

(54) APPARATUSES FOR PERFORMING RAPID DIAGNOSTIC TESTS

(71) Applicant: Detect, Inc., Guilford, CT (US)

(72) Inventors: Todd Roswech, Ivoryton, CT (US); Matthew Dyer, Spring, TX (US)

(21) Appl. No.: 17/525,194

(22) Filed: Nov. 12, 2021

Related U.S. Application Data
(60) Provisional application No. 63/113,748, filed on Nov. 13, 2020.

(51) Int. Cl. G01N 33/543; B01L 3/00; B01L 7/00

(52) U.S. Cl. CPC G01N 33/54388; B01L 3/5029; B01L 7/00; B01L 3/523; B01L 3/565; B01L 2300/123; B01L 2400/0481; B01L 2300/069; B01L 2300/042; B01L 2400/0487; B01L 2300/044

(57) ABSTRACT

Diagnostic devices for performing diagnostic tests are provided, as well as methods that utilize the diagnostic devices, methods for manufacturing the diagnostic devices, and test kits for performing the diagnostic tests. The diagnostic devices include a sample chamber with an opening for receiving a sample; a fluid chamber containing a fluid; and a test and readout chamber containing a lateral-flow assay (LFA) strip. The fluid chamber and/or the test and readout chamber is/are burstable and is/are configured to be in fluid connection with the sample chamber upon bursting. The fluid chamber may be flexible and configured to burst at a seal separating the sample chamber from the fluid chamber. The seal maybe configured to break when a bursting force is applied to the fluid chamber.

### CROSS-REFERENCE TO RELATED APPLICATIONS

The present application claims the benefit of priority of U.S. Provisional Application No. 63/113,748 filed Nov. 13, 2020, entitled “APPARATUSES FOR PERFORMING RAPID DIAGNOSTIC TESTS” (Attorney Docket No. H0966.70051US00), the entire contents of which is incorporated by reference herein.

### FIELD

The technology of the present invention relates generally to test apparatuses, test kits, and methods of using the test apparatuses and/or the test kits to perform rapid diagnostic tests to detect the presence of one or more target nucleic-acid sequences.

### BACKGROUND

The ability to rapidly diagnose diseases—particularly highly communicable infectious diseases—is critical to preserving human health through early detection and containment of the infectious diseases until reliable preventive measures (e.g., vaccines) and/or medicinal treatments or cures are developed. Rapid testing is critical to determining infected individuals quickly and minimizing their interactions with others, in order to minimize the spread of the diseases. As one example, the high level of contagiousness, the high mortality rate, and the lack of an early treatment or vaccine for the coronavirus disease 2019 (COVID-19) have resulted in a pandemic that has already infected millions and killed hundreds of thousands of people. The existence of rapid, accurate diagnostic tests, useable for detecting COVID-19 as well as other diseases, could allow individuals infected with a disease to be quickly identified and isolated, which could assist with containment of the disease. In the absence of such diagnostic tests, diseases such as COVID-19 may spread unchecked throughout communities.

### SUMMARY

Provided herein are apparatuses and techniques for performing diagnostic tests useful for detecting one or more pathogens by detecting one or more target nucleic-acid sequences corresponding to the pathogens. The apparatuses and techniques described herein may be performed in a point-of-care (POC) setting or home setting by a lay person without specialized equipment and without training in laboratory procedures.

According to an aspect of the present technology, a diagnostic apparatus for performing a rapid diagnostic test is provided. The apparatus may be comprised of: a sample chamber configured with an opening through which a sample is received in the sample chamber; a first fluid chamber containing a first fluid; and a test and readout chamber containing a lateral-flow assay (LFA) strip. The first fluid chamber and/or the test and readout chamber may be burstable and may be in fluid connection with the sample chamber upon bursting.

In some embodiments of this aspect, the first fluid chamber may be a flexible first fluid chamber and may be configured to burst a burstable first seal. The first seal may separate the sample chamber from the first fluid chamber. The first seal may be configured to burst when a first bursting force is applied to the first fluid chamber.

In some embodiments of this aspect, the apparatus may further be comprised of a burstable second fluid chamber containing a second fluid. The second fluid chamber may be configured to be in fluid connection with the sample chamber upon bursting. In some embodiments, the second fluid chamber may be a flexible second fluid chamber and may be configured to burst a burstable second seal separating the sample chamber from the second fluid chamber. The second seal may be configured to burst when a second bursting force is applied to the second fluid chamber. In some embodiments, the test and readout chamber may be a flexible chamber and may be configured to burst a burstable third seal separating the sample chamber from the test and readout chamber. The third seal may be configured to burst when the second bursting force is applied to the second fluid chamber and/or when a third bursting force is applied to the test and readout chamber.

In some embodiments of this aspect, the apparatus may further be comprised of a conduit connecting the sample chamber and the test and readout chamber. An intake end of the LFA strip may be disposed at an outlet end of the conduit.

In some embodiments of this aspect, the test and readout chamber may be comprised of a window that enables a test region of the LFA strip to be visible through the window.

In some embodiments of this aspect, the apparatus may further be comprised of a sample swab having a cap end and a sample end. The cap end of the sample swab may be configured to seal the opening of the sample chamber. The sample end of the sample swab may be configured to extend into a base portion of the sample chamber to deliver the sample into the base portion.

In some embodiments of this aspect, the first chamber may be configured to burst at a base end of the first chamber. The sample chamber may be configured to have an upright position such that, upon bursting, gravity causes the first fluid to flow outward from the base end of the first chamber into the sample chamber.

In some embodiments of this aspect, the apparatus may further be comprised of a heater configured to heat the sample chamber.

According to another aspect of the present technology, a rapid diagnostic test apparatus is provided. The apparatus may be comprised of a container configured to receive a sample in an internal cavity. The container may be comprised of: a rupturable first compartment holding a first fluid and configured to be in fluid communication with the internal cavity upon rupturing; and a lateral-flow assay (LFA) strip disposed in a portion of the container.

In some embodiments of this aspect, the container may be resealable and may have an opened position in which the internal cavity of the container is accessible to receive the sample, and a closed position in which the internal cavity is not accessible. In some embodiments, the container may further be comprised of: a rupturable second compartment holding a second fluid and configured to be in fluid communication with the internal cavity upon rupturing, and a rupturable third compartment holding the LFA strip and configured to be in fluid communication with the internal cavity upon rupturing. In some embodiments, at least one of the first, second, and third compartments is comprised of a burstable seal configured to rupture upon application of a rupturing force. The rupturing force may be comprised of any one or any combination of: a squeezing force, a pinching force, a jabbing force, a rubbing force, and a bending force. In some embodiments, the first compartment may be configured to rupture into the internal cavity such that the first fluid flows into the internal cavity, and the second compartment also may be configured to rupture into the internal cavity such that the second fluid flows into the internal cavity.

In some embodiments of this aspect, the apparatus may further be comprised of a first force applicator movably attached to the container and configured to apply a first bursting force to the first compartment.

In some embodiments of this aspect, the apparatus may further be comprised of a manifold configured to receive a base portion of the container such that, when the container is in a mounted position on the manifold, the manifold enables the first fluid to flow from the first compartment to the internal cavity.

According to another aspect of the present technology, a test kit for performing a rapid diagnostic test is provided. The test kit may be comprised of: a diagnostic test apparatus, which may include a fluid compartment containing a fluid for a test procedure, and a test compartment containing a lateral-flow assay (LFA) strip; and a sample swab configured to collect a sample for the test procedure. The fluid compartment and/or the test compartment or may be burstable.

In some embodiments of this aspect, the test kit may further be comprised of a heater configured to heat at least a portion of the diagnostic test apparatus.

In some embodiments of this aspect, the test kit may further be comprised of a reagent to be used in the test procedure.

### BRIEF DESCRIPTION OF THE DRAWINGS

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way.

FIGS. 1A through 1C schematically depict a diagnostic device with burstable chambers holding fluids for a rapid diagnostic test procedure, according to some embodiments of the present technology.

FIG. 2 schematically depicts a diagnostic device with burstable chambers holding fluids for a rapid diagnostic test procedure, according to some embodiments of the present technology.

FIG. 3 schematically depicts a diagnostic device with burstable compartments and a cap-type resealable container, according to some embodiments of the present technology.

FIGS. 4A and 4B each schematically depict a diagnostic device with burstable compartments and a zipper-type resealable container, according to some embodiments of the present technology.

FIGS. 5A through 5E schematically depict a diagnostic device with burstable compartments and a manifold, according to some embodiments of the present technology.

FIGS. 6A through 6C show flow diagrams of methods of using diagnostic devices with burstable compartments, according to some embodiments of the present technology.

FIG. 7 shows a flow diagram of a method of manufacturing a diagnostic device, according to some embodiments of the present technology.

### DETAILED DESCRIPTION

### 1. Introduction

The present disclosure provides test apparatuses, test kits, and methods of using the test apparatuses and/or the test kits (collectively referred to as “diagnostic systems” herein) for performing, in a clinical environment (e.g., medical facility, laboratory, etc.) and/or in a non-clinical environment (e.g., a home, a business office, a school, etc.), rapid diagnostic testing to detect one or more target nucleic-acid sequences. The diagnostic systems described herein, according to some embodiments of the present technology, may be self-administrable and may be comprised of any combination of: a sample-collecting device (e.g., a swab), reagents, a diagnostic device that enables a reaction between the reagents and a sample, and a detection component, which may be included as part of the diagnostic device.

According to some embodiments of the present technology, the sample-collecting device may be a disposable swab configured to contact a test subject to collect the sample and to transfer the collected sample to the diagnostic device, and then may be discarded. In some other embodiments, the sample-collecting device may comprise part of the diagnostic device and may participate in a procedure of the test. For example, the sample-collecting component may facilitate an interaction between the sample and one or more of the reagents.

According to some embodiments of the present technology, the detection component may be an assay vehicle (e.g., a strip) on which is contained or attached one or more reagents for detecting the presence of a target nucleic-acid sequence indicative of a particular pathogen or disease. In some embodiments, the assay vehicle may contain or have attached thereto a plurality of reagents for detecting the presence of a plurality of different target nucleic-acid sequences indicative of a plurality of different pathogens or diseases. In some embodiments, the assay vehicle may be a lateral-flow assay (LFA) strip configured to come into contact with a sample solution and to enable the sample solution to flow through the strip from one end to another. Observable changes in a region of the LFA strip may indicate the presence of the target nucleic-acid sequence, indicating that the test subject may be afflicted with the pathogen or disease corresponding to the target nucleic-acid sequence. In some instances, for LFA strips that are able to detect more than one pathogen or disease, observable changes in multiple regions of the LFA strip may indicate the presence of multiple target nucleic-acid sequences, indicating that the test subject may be afflicted with more than one pathogen or disease corresponding to the target nucleic-acid sequences. In some embodiments, the detection component may be incorporated in the diagnostic device to, for example, minimize handling by a user, who may be a person without medical training. For example, the diagnostic device may be comprised of a window that may enable changes in the assay vehicle to be visible, which may enable a user to perform a reading of a test result and/or may enable an image (e.g., a photograph) of the assay vehicle to be captured and automatically read or analyzed by a computer algorithm.

According to some embodiments of the present technology, the reagents may be comprised of any one or any combination of: one or more lysis reagents, one or more nucleic-acid amplification reagents, and one or more CRISPR/Cas detection reagents. The reagents may be in solid form (e.g., lyophilized, crystallized, etc.) and therefore, in some embodiments, included with the reagents may be one or more buffer solutions configured to activate one or more of the reagents. Additionally, included with the reagents may be one or more diluent fluids for achieving a desirable concentration of reagent fluids during various procedures of the test.

According to some embodiments of the present technology, the diagnostic device may comprise components for handling the reagents prior to their use in the test, components for storing and/or handling the reagents or the sample, or mixtures thereof, during various procedures of the test, and components for promoting reactions between the sample and one or more of the reagents. For example, such components may include one or more burstable chambers or compartments holding one or more reagents and/or one or more reaction fluids and/or an LFA strip. (The terms “burstable chamber” and “burstable compartment” may be used interchangeable herein.) In some embodiments, such compartments may be configured to burst to enable the one or more reagents to react with the one or more reaction fluids and the sample, to form a sample fluid. In some embodiments, each of the burstable compartments may include a burstable seal (e.g., a frangible seal) configured to break when a rupturing force is applied to the burstable compartment. In some embodiments, the rupturing force may be applied by a user directly. For example, the user may, e.g., squeeze or

pinch a portion of a burstable compartment to cause pressure within the burstable compartment to exceed a threshold pressure at which a wall of the burstable compartment and/or a burstable seal of the burstable compartment breaks. In some embodiments, the rupturing force may be applied indirectly by a user via a mechanical or electromechanical force applicator. For example, the user may cause the force applicator to move against a burstable compartment to, e.g., squeeze or pinch the burstable compartment to cause pressure within the burstable compartment to exceed the threshold pressure.

### 2. “Burstable-Type” Test Systems, Components, and Methods

2.1 Diagnostic Devices with Burstable Chambers Attached to Sample Chamber

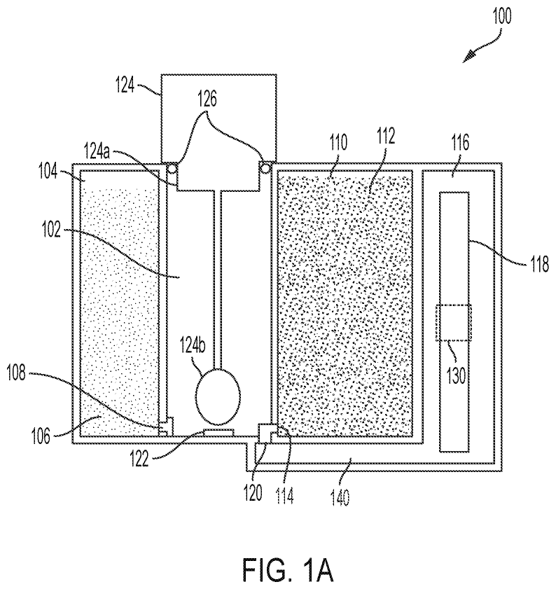

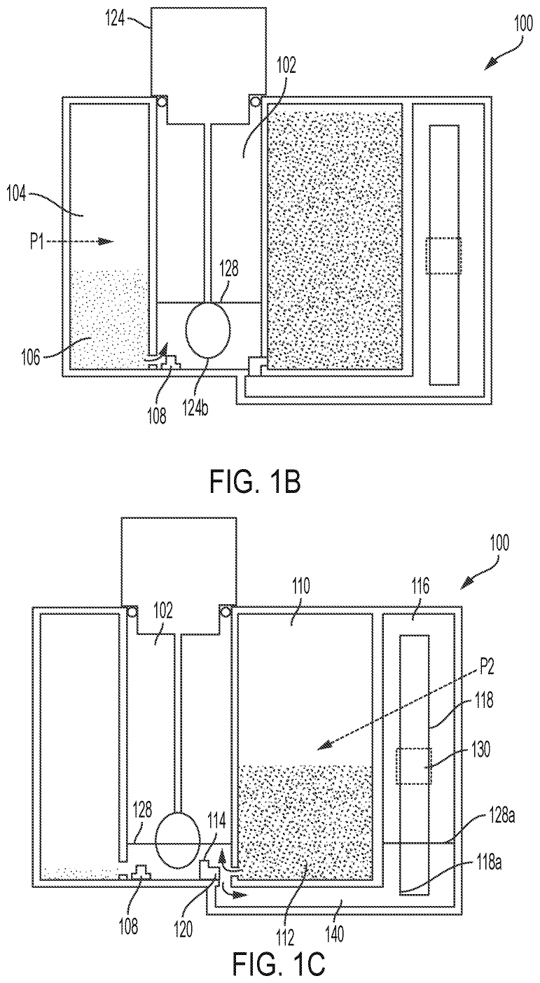

FIG. 1A schematically depicts a diagnostic device 100 comprised of burstable chambers holding fluids to be used in a rapid diagnostic test procedure, according to some embodiments of the present technology. The diagnostic device 100 may be comprised of a sample chamber 102 configured with an opening through which a sample may be received in the sample chamber 102. A first fluid chamber 104 may be connected to the sample chamber 102 and may contain a first fluid 106 therein. A burstable first seal 108 may separate the first fluid 106 from the sample chamber 102. The first fluid chamber 104 may be arranged adjacent the sample chamber 102, as shown, or may be connected to the sample chamber 102 via a conduit. A second fluid chamber 110 may be connected to the sample chamber 102 and may contain a second fluid 112 therein. A burstable second seal 114 may separate the second fluid 112 from the sample chamber 102. The second fluid chamber 110 may be arranged adjacent the sample chamber 102, as shown, or may be connected to the sample chamber 102 via a conduit. A test and readout chamber 116 may be separated from the sample chamber 102 by a burstable third seal 120. A LFA strip 118 may be housed in the test and readout chamber 116. In some embodiments, the test and readout chamber 116 may be separated from the sample chamber 102 by the third seal 120. In some embodiments, the test and readout chamber 116 may be separated from the sample chamber 102 by a conduit 140 in addition to the third seal 124, as shown.

According to some embodiments of the present technology, the sample chamber 102 may contain a reagent 122. During transport or storage of the diagnostic device 100, a removable cap (not shown) may cover an opening of the sample chamber 102 to prevent contamination of the reagent 122. For instance, the cap may seal against a sealing surface (e.g., an o-ring or rubber gasket) 126 at the opening of the sample chamber 102. Although the reagent 122 is schematically depicted to be a solid (e.g., a lyophilized reagent), in some embodiments the reagent 122 may be in the form of a fluid.

FIG. 1A schematically depicts the diagnostic device 100 after a sample has been introduced to the sample chamber 102, according to some embodiments of the present technology. In some embodiments, the sample may be provided to the sample chamber 102 via a sample swab 124. The sample swab may be comprised of a cap end 124a configured to seal against the sealing surface 126 at the opening of the sample chamber 102, as shown. The sample swab 124 also may be comprised of a sample end 124b configured to extend into a base portion of the sample chamber 102 to deliver the sample into the base portion of the sample chamber 102.

According to some embodiments of the present technology, the sample chamber 102 may be formed of a rigid material (e.g., metal, glass, a hard plastic, etc.). The first and second fluid chambers 104, 110 may be attached to an external surface of the sample chamber 102, and the first and second seals 108, 114 may be located at the base portion of the sample chamber 102. With such an arrangement, when the sample chamber 102 is in an upright position and pressure is applied to the first liquid 106 causing pressure against the first seal 108 to exceed a threshold, the first seal 108 may burst and be pushed into the sample chamber 102. Gravity may then cause the first fluid 106 to flow downward and out of the first fluid chamber 104 into the sample chamber 102. Similarly, when the sample chamber 102 is in an upright position and pressure is applied to the second liquid 112 causing pressure against the second seal 114 to exceed a threshold, the second seal 114 may burst and be pushed into the sample chamber 102. Gravity may then cause the second fluid 112 to flow downward and out of the second fluid chamber 110.

According to some embodiments of the present technology, some or all of the first fluid chamber 104 may be formed of a flexible material (e.g., a resilient material), and the first seal 108 may be configured to burst or rupture when a first rupturing force P1 is applied to the first fluid chamber 104 to deform the first fluid chamber 104, as schematically depicted in FIG. 1B. Deformation of the first fluid chamber 104 may increase the pressure of the first liquid 106 against the first seal 108, causing the first seal 108 to burst. As schematically depicted in FIG. 1B, after the first seal 108 is ruptured, the first fluid 106 may flow into the sample chamber 102 and cause a fluid level 128 to rise in the sample chamber 102. For example, the first fluid chamber 104 may be a first pouch (e.g., a flexible metal foil pouch, a resilient polymer pouch, a squeezable bladder, etc.), and the first rupturing force P1 may be a squeezing force or a pinching force applied to the first pouch 104 by a user. The first seal 108 may be a plug seal or an adhesive seal or any seal configured to burst or rupture when a force against the first seal 108 exceeds the first rupturing force P1.

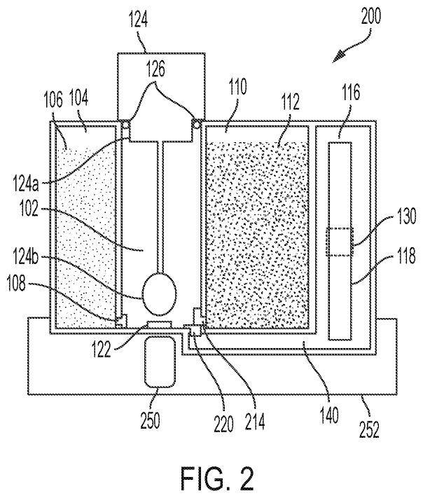

According to some embodiments of the present technology, the reagent 122 may be a lyophilized reagent, and the first fluid 106 may be a buffer fluid configured to activate the lyophilized reagent 122 to form a reagent solution. When the first rupturing force P1 is applied to the first fluid chamber 104 to cause the first seal 108 to rupture, the buffer fluid 106 may flow into the sample chamber 102 to activate the lyophilized reagent 122 to form the reagent solution. When the fluid level 128 of the reagent solution in the sample chamber 102 is sufficient to contact the sample on the sample end 124b of the sample swab 124, interaction of the sample with the reagent solution may form a sample fluid. For example, in a case where the reagent 122 is a lyophilized amplification reagent, the buffer fluid 106 may activate the amplification reagent 122 to form an amplification solution that amplifies the sample to form the sample fluid. In some embodiments, the sample and the reagent solution may be heated to form the sample fluid, as discussed below in connection with FIG. 2. It should be appreciated that although the sample is depicted to be provided via the sample swab 124, in some embodiments the sample may be provided via other means (e.g., a dropper, a sample stick that remains in the sample chamber 102 but is not attached to a cap, a sample stick that is inserted through the opening in the sample chamber 102 and swirled in the reagent solution in the sample chamber 102 but is removed from the sample chamber 102 after swirling, etc.).

According to some embodiments of the present technology, the second seal 114 may be configured to rupture or burst when a second rupturing force P2 is applied to the second fluid chamber 110, as schematically depicted in FIG. 1C. After the second seal 114 is ruptured, the second fluid 112 may flow into the sample chamber 102 and mix with the sample fluid. For example, the second fluid chamber 110 may be a second pouch (e.g., a flexible metal foil pouch, a resilient polymer pouch, a squeezable bladder, etc.), and the second rupturing force P2 may be a squeezing force or a pinching force applied to the second pouch 110 by the user. The second seal 114 may be a plug seal or an adhesive seal or any seal configured to burst when a force against the seal 114 exceeds the second rupturing force P2. In some embodiments, the second fluid 112 may be a diluent fluid configured to dilute the sample fluid to form a diluted sample fluid. When the second rupturing force P2 is applied to the second fluid chamber 110 to cause the second seal 114 to rupture, the diluent fluid may flow out of the second fluid chamber 110 into the sample chamber 102 to mix with the sample fluid to form the diluted sample fluid in the sample chamber 102.

According to some embodiments of the present technology, the third seal 120 may be configured to rupture or burst when the second rupturing force P2 is applied to the second fluid chamber 110. This may occur, for example, in cases where the second and third seals 114, 120 are connected to each other and rupture or burst together. The sample fluid may flow out of the sample chamber 102 and the diluent fluid 112 may flow out of the second fluid chamber 110 with one application of the second rupturing force P2, and the sample fluid and the diluent fluid 112 may mix to form the diluted sample fluid in the test and readout chamber 116 and/or in the conduit 140 leading to the test and readout chamber 116. In some embodiments, the conduit 140 may be configured such that an intake end 118a of the LFA strip 118 may be disposed at an outlet end of the conduit 140. The diluted sample fluid may be absorbed at the intake end 118a of the LFA strip 118 first and then conveyed to test regions of the LFA strip 118 via capillary action. In some embodiments, a fluid level 128a of the diluted sample fluid in the test and readout chamber 116 may be sufficient to contact the intake end 118a of the LFA strip 118 directly but may not be sufficient to contact the test regions of the LFA strip 118 directly, thus requiring the diluted sample fluid to reach the test regions via capillary action.

According to some embodiments of the present technology, the test and readout chamber 116 may be comprised of a window 130 through which the test regions of the LFA strip 118 are visible. In some embodiments, the test regions may be readable through the window 130 by the user and/or by an electronic reader (e.g., a smartphone camera). For example, the electronic reader may provide image data to a software application configured to analyze the image data and to output an analysis result (e.g., a presence or an absence of a pathogen in the sample). Optionally, the electronic reader may be configured to upload the image data to an external analysis system via, e.g., the Internet.

FIG. 2 schematically depicts a diagnostic device 200 that is a variation of the diagnostic device 100, according to some embodiments of the present technology. Aspects of the diagnostic devices 100, 200 that are the same or similar will have the same reference numerals, and their descriptions will not be repeated for the diagnostic device 200. Similar to the diagnostic device 100, the diagnostic device 200 utilizes burstable chambers to hold fluids for a test procedure. FIG. 2 schematically depicts the diagnostic device 200 after a sample has been introduced to the sample chamber 102. A difference in the diagnostic device 200 relative to the diagnostic device 100 may be seen in the second fluid chamber 110 of the diagnostic device 200, where a burstable second seal 214 may separate the second fluid 112 in the second fluid chamber 110 from the sample chamber 102. Another difference may be seen in the test and readout chamber 116 of the diagnostic device 200, where a burstable third seal 220 may separate the sample chamber 102 from the LFA strip 118 in the test and readout chamber 116. As schematically shown in FIG. 2, the second seal 214 and the third seal 220 may be physically distinct objects. In some embodiments, the second seal 214 and may be configured to burst or rupture when a rupturing force (e.g., P2) is applied to the second fluid chamber 110 of the diagnostic device 200. If the second seal 214 is ruptured but the third seal 220 remains in place and is not ruptured, the second fluid 112 may flow into the sample chamber 102 and mix with the sample fluid. For example, the second fluid 112 may be the diluent fluid described above and may mix with the sample fluid to form the diluted sample fluid. In some embodiments, the third seal 220 and may be configured to burst or rupture when a rupturing force (not shown) is applied to the test and readout chamber 116 of the diagnostic device 200. If the third seal 220 is ruptured but the second seal 214 remains in place and is not ruptured, the sample fluid (undiluted) may flow out of the sample chamber 102 towards and the test and readout chamber 116. If both the second seal 214 and the third seal 220 are ruptured, the sample fluid may flow out of the sample chamber 102 and the second (diluent) fluid 112 may flow out of the second fluid chamber 110, and the sample fluid and the diluent fluid 112 may mix to form the diluted sample fluid in the test and readout chamber 116 and/or in the conduit 140 leading to the test and readout chamber 116.

According to some embodiments of the present technology, a heater 250 may be used to heat the sample chamber 102 (e.g., to heat the sample fluid). The heater 250 may be incorporated in a housing 252 configured to hold the diagnostic device 100 or the diagnostic device 200. For example, the housing 252 may have a recess configured to receive a part of the sample chamber 102, or the housing 252 may be configured with a platform on which the sample chamber 102 may sit. In some embodiments, the housing 252 and/or the heater 250 may include a sensor (not shown) configured to sense a presence of the diagnostic device 100 or the diagnostic device 200. For example, the sensor may sense when the sample chamber 102 is in a heating position on the heater 250. The heater 250 may be configured to perform a heating procedure automatically when the sensor detects the sample chamber 102 in the heating position.

According to In some embodiments of the present technology, a rapid diagnostic test kit may include any one or any combination of: the diagnostic device 100 or the diagnostic device 200, with a removable cap covering the sample chamber 102; the reagent 122, which may be provided in the sample chamber 102 or in a separate package to be added to the sample chamber (e.g., a reagent carrier described in US Patent Application Publication No. 2021/0291177 A1 entitled “Reagent Carrier for Rapid Diagnostic Tests,” which is incorporated by reference herein); the sample swab 124; the heater 250, which may be incorporated in the housing 252; software for electronically reading the LFA strip 118; and instructions (in electronic form and/or in paper form) for using the test kit. Components of the test kit may be packaged individually or together.

2.2 Diagnostic Devices with Burstable Compartments and Resealable Container

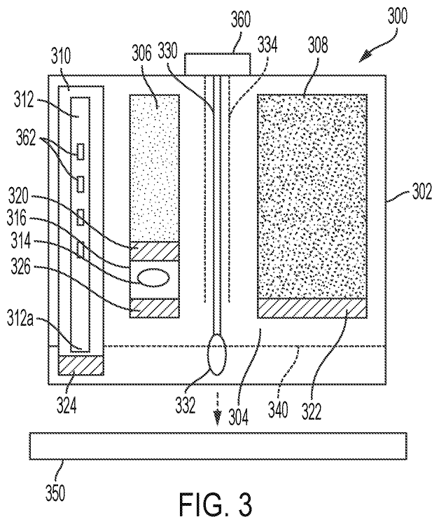

FIG. 3 schematically depicts a diagnostic device 300 for performing a rapid diagnostic test procedure, according to some embodiments of the present technology. The diagnostic device 300 may be comprised of a resealable container 302 that includes at least one rupturable compartment holding a test material for the test procedure. A LFA strip 312 may be disposed in the container 302. The container 302 may be unsealed to an opened position to enable an internal cavity 304 of the container 302 to be accessed (e.g., to provide a sample to the internal cavity 304), and may be sealed to a closed position to prevent contamination of the internal cavity 304 and/or to prevent access to the internal cavity 304.

According to some embodiments of the present technology, the container 302 may be configured with a guide channel 334 configured to receive the sample via a sample swab 330. The guide channel 334 may be configured to guide the sample swab 330 to, e.g., a bottom or base region of the internal cavity 304. The bottom or base region may be a region to which fluid in the internal cavity 304 flows due to gravity, when the container 302 is in an upright position. For example, the guide channel 334 may be formed of an open-ended tube through which the sample swab 330 may be inserted. In some embodiments, the sample swab 330 may have a length such that the sample, which may be carried by a sample element 332 at an end of the sample swab 330, may reach a desired location in the internal cavity 304 when the container 302 is sealed with the sample swab 330 inside the internal cavity 304.

According to some embodiments of the present technology, the container 302 may be comprised of a rupturable first compartment 306 holding a first fluid. The first compartment 306 may be configured to be in fluid communication with the internal cavity 304 upon rupturing. The container 302 also may be comprised of a rupturable second compartment 308 holding a second fluid. The second compartment 308 may be configured to be in fluid communication with the internal cavity 304 upon rupturing. The LFA strip 312 may be held in a rupturable third compartment 310 of the container 302. The third compartment 310 may be configured to be in fluid communication with the internal cavity 304 upon rupturing.

According to some embodiments of the present technology, the first fluid may be a reagent fluid (e.g., an amplification fluid). Upon rupturing of the first compartment 306, the reagent fluid may be released from the first compartment 306 and may flow into the internal cavity 304 to interact with the sample to form a sample fluid.

According to some embodiments of the present technology, the container 302 may be comprised of a lyophilized reagent 314 held in a rupturable fourth compartment 316 of the container 302. The first fluid may be a buffer fluid configured to activate the lyophilized reagent 314. Upon rupturing of the first compartment 306 and the fourth compartment 316, the reagent 314 and the buffer fluid may be released into the internal cavity 304 to interact with the sample carried by the sample element 332 to form a sample fluid.

According to some embodiments of the present technology, the container 302 may be comprised of a lyophilized reagent held in the internal cavity 304 the container 302 or added to the internal cavity 304 during the test procedure, and the first fluid may be a buffer fluid configured to activate the lyophilized reagent. Upon rupturing of the first compartment 306, the buffer fluid may be released into the internal cavity 304 to interact with the reagent and the sample carried by the sample element 332 to form a sample fluid.

According to some embodiments of the present technology, the second fluid may be a diluent fluid. Upon rupturing of the second compartment 308, the diluent fluid may be released from the second compartment 308 and may flow into the internal cavity 304 to interact with the sample fluid to form a diluted sample fluid. In some embodiments, upon rupturing the third compartment 310, the LFA strip 312 in the third compartment 310 may be exposed to the diluted sample fluid. For example, the third compartment 310 may rupture to form a hole (not shown) that may enable fluid communication between the internal cavity 304 and the third compartment 310. A level 340 of the diluted sample fluid in the internal cavity 304 may be sufficient to reach the hole in the third compartment 310 when the container 302 is in the upright position. In some embodiments, the hole may be formed at a burstable seal 324 of the third compartment 310. An intake end 312a of the LFA strip 312 may disposed proximate the hole at the burstable seal 324. The diluted sample fluid may be absorbed at the intake end 312a of the LFA strip 312 and may be conveyed to test regions 362 of the LFA strip 312 via capillary action. In some embodiments, the third compartment 310 may be comprised of a window (not shown) through which the LFA strip 312 is visible, such that the test regions 362 of the LFA strip 312 may be readable by a human reader and/or an electronic reader through the window.

According to some embodiments of the present technology, any one or any combination of the first compartment 306, the second compartment 308, the third compartment 310, and the fourth compartment 316 may be attached to a surface of the internal cavity 304. For example, the first compartment 306 may be configured to rupture into the internal cavity 304 such that the first fluid flows into the internal cavity 304, and the second compartment 308 may be configured to rupture into the internal cavity 304 such that the second fluid flows into the internal cavity 304. In some embodiments, any one or any combination of the first, second, third, and fourth compartments 306, 308, 310, 316 may be configured to rupture upon application of a rupturing force. For example, the rupturing force may be comprised of any one or any combination of: a squeezing force, a pinching force, a jabbing force, a rubbing force, and a bending force.

According to some embodiments of the present technology, the first compartment 306 may be comprised of a first burstable seal 320, the second compartment 308 may be comprised of a second burstable seal 322, the third compartment 310 may be comprised of the third burstable seal 324, and the fourth compartment 316 may be comprised of a fourth burstable seal 326. In some embodiments, any one or any combination of the burstable seals 320, 322, 324, 326 may be configured to rupture upon application of a rupturing force. For example, the rupturing force may be comprised of any one or any combination of: a squeezing force, a pinching force, a jabbing force, a rubbing force, and a bending force.

According to some embodiments of the present technology, the container 302 may be comprised of a flexible polymeric bag supporting the first, second, third, and fourth compartments 306, 308, 310, 316. For example, the flexible polymeric bag may be comprised of a high-density polyethylene (HDPE) material. In some embodiments, the internal cavity 304 of the container 302 may be sealable with a removeable cap 360. The cap 360 may be configured to provide a leak-tight seal when covering an opening of the container 302 leading to the internal cavity 304, and to provide access to the internal cavity 304 when not covering the opening. In some embodiments, the cap 360 may be a screw-on/off cap or a friction-fit cap. In some embodiments, the sample swab 330 may be integrated with and extend from the cap 360, as depicted in FIG. 3. In some embodiments, the diagnostic device 300 may be shipped and/or stored with a disposable cap (not shown), which may be replaced with the cap 360 integrated with the sample swab 330 when the apparatus 300 is used in a test procedure, with the sample swab 330 being used to deliver a sample into the internal cavity 304 of the container 302.

According to some embodiments of the present technology, the diagnostic device 300 may be comprised of a heater 350 configured to heat the container 302 (e.g., to heat the sample fluid prior to dilution with the diluent fluid). In some embodiments, the heater 350 may be attached to an external surface of the container 302 opposite the bottom or base region of the internal cavity 304. In some other embodiments, the heater 350 may be a separate unit configured to support and heat the container 302 when the container 302 is placed on the heater 350.

According to some embodiments of the present technology, a rapid diagnostic test kit may include any one or any combination of: the diagnostic device 300, with or without a the removable cap 360 covering the internal cavity 304 of the container 302; the sample swab 330, which may extend from the cap 360 or may not be attached to a cap; the heater 350; software for electronically reading the LFA strip 312; and instructions (in electronic form and/or in paper form) for using the test kit. Components of the test kit may be packaged individually or together.

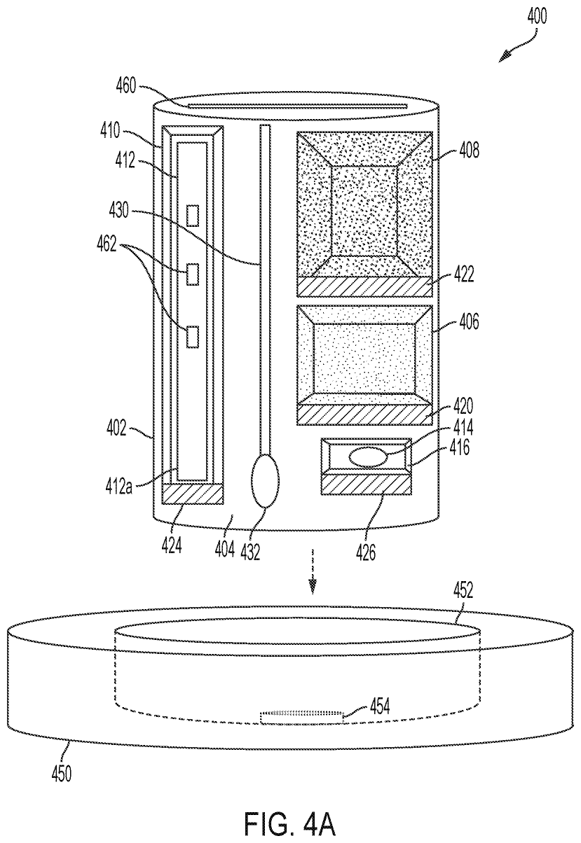

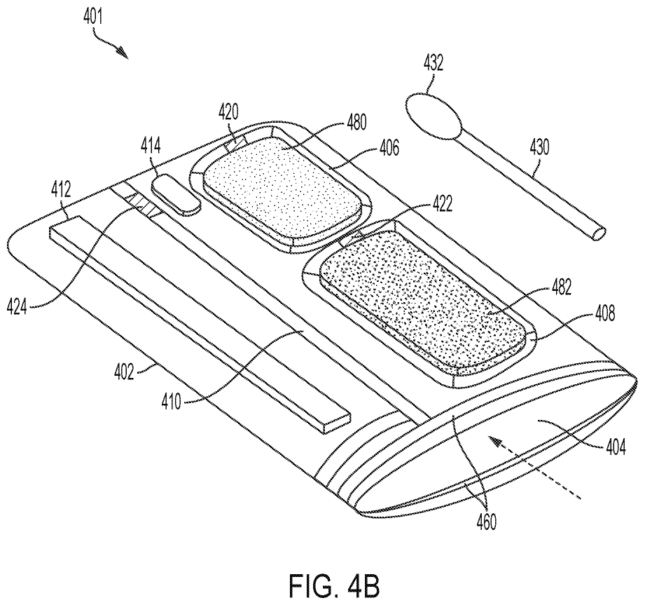

FIG. 4A schematically depicts a diagnostic device 400 for performing a rapid diagnostic test procedure, according to some embodiments of the present technology. The diagnostic device 400 may be comprised of a resealable container 402 that includes at least one rupturable compartment holding a test material for the test procedure. A LFA strip 412 may be disposed in the container 402. The container 402 may be unsealed to an opened position to enable an internal cavity 404 of the container 402 to be accessed (e.g., to provide a sample to the internal cavity 404), and may be sealed to a closed position to prevent contamination of the internal cavity 404 and/or to prevent access to the internal cavity 404.

According to some embodiments of the present technology, the container 402 may be configured to receive the sample via a sample element 432 of a sample swab 430. Although not shown in FIG. 4A, a guide channel may be provided to guide the sample swab 430 to, e.g., a bottom or base region of the internal cavity 404. In some embodiments, the sample swab 430 may have a length that enables the sample swab 430 to be sealed inside the internal cavity 404. The container 402 may be comprised of a rupturable first compartment 406 holding a first fluid. The first compartment 406 may be configured to be in fluid communication with the internal cavity 404 upon rupturing. The container 402 also may be comprised of a rupturable second compartment 408 holding a second fluid. The second compartment 408 may be configured to be in fluid communication with the internal cavity 404 upon rupturing. The LFA strip 412 may be held in a rupturable third compartment 410 of the container 402. The third compartment 410 may be configured to be in fluid communication with the internal cavity 404 upon rupturing.

According to some embodiments of the present technology, the container 402 may be comprised of a lyophilized reagent 414 held in a rupturable fourth compartment 416 of the container 402. The first fluid may be a buffer fluid configured to activate the reagent 414. Upon rupturing of the first compartment 406 and the fourth compartment 416, the reagent 414 and the buffer fluid may be released into the internal cavity 404 to interact with a sample carried by a swab element 432 of the sample swab 430, to form a sample fluid. In some embodiments, the second fluid may be a diluent fluid. Upon rupturing of the second compartment 408, the diluent fluid may be released from the second compartment 408 and may flow into the internal cavity 404 to interact with the sample fluid to form a diluted sample fluid. In some embodiments, upon rupturing of the third compartment 410, the LFA strip 412 in the third compartment 410 may exposed to the diluted sample fluid. For example, the third compartment 410 may rupture to form a hole (not shown) that enables fluid communication between the internal cavity 404 and the third compartment 410. A level of the diluted sample fluid in the internal cavity 404 may be sufficient to reach the hole in the third compartment 410 when the container 402 is in the upright position. In some embodiments, the hole may be formed at a burstable seal 424 of the third compartment 410. An intake end 412a of the LFA strip 412 may disposed proximate the hole at the burstable seal 424. The diluted sample fluid may be absorbed at the intake end 412a and may be conveyed to test regions 462 of the LFA strip 412 via capillary action. In some embodiments, the third compartment 410 may be comprised of a window (not shown) through which the LFA strip 412 is visible, such that the test regions 462 may be readable by a human reader and/or an electronic reader through the window.

According to some embodiments of the present technology, any one or any combination of the first compartment 406, the second compartment 408, the third compartment 410, and the fourth compartment 416 is or are attached to a surface of the internal cavity 404. For example, the first compartment 406 may be configured to rupture into the internal cavity 404 such that the first fluid flows into the internal cavity 404, and the second compartment 408 may be configured to rupture into the internal cavity 404 such that the second fluid flows into the internal cavity 404. In some embodiments, any one or any combination of the first, second, third, and fourth compartments 406, 408, 410, 416 may be configured to rupture upon application of a rupturing force. For example, the rupturing force may be comprised of any one or any combination of: a squeezing force, a pinching force, a jabbing force, a rubbing force, and a bending force.

According to some embodiments of the present technology, the first compartment 406 may be comprised of a burstable first seal 420, the second compartment 408 may be comprised of a burstable second seal 422, the third compartment 410 may be comprised of the burstable third seal 424, and the fourth compartment 416 may be comprised of a burstable fourth seal 426. In some embodiments, any one or any combination of the seals 420, 422, 424, 426 may be configured to rupture upon application of a rupturing force. For example, the rupturing force may be comprised of any one or any combination of: a squeezing force, a pinching force, a jabbing force, a rubbing force, and a bending force.

According to some embodiments of the present technology, the container 402 may be comprised of a flexible polymeric bag supporting the first, second, third, and fourth compartments 406, 408, 410, 416. For example, the flexible polymeric bag may be comprised of HDPE. In some embodiment, the container 402 may be equipped with a zipper-type sealing device 460 configured to provide a leak-tight seal of the internal cavity 404 when in a zipped-shut state, as depicted in FIG. 4A. The internal cavity 404 may be accessed when the sealing device 460 is in a zipped-open state.

According to some embodiments of the present technology, the diagnostic device 400 may be comprised of a heater 450 configured to heat at least a portion of the container 402 (e.g., to heat the sample fluid prior to dilution with the diluent fluid). For example, a base of the container 402 may be configured to be received in a recess 452 the heater 450. In some embodiments, the heater 450 may be configured to perform a heating procedure automatically when base of the container 402 is detected to be in the recess 452 of the heater 450. For example, a sensor 454 (e.g., an optical detector) may be provided on the heater 450 (e.g., in the recess 452) to detect when the base of the container 402 is seated in the recess 452.

FIG. 4B schematically depicts a diagnostic device 401 that is a variation of the diagnostic device 400 described above and depicted in FIG. 4A. Aspects of the diagnostic device 401 that may be similar to or the same as those of the diagnostic device 400 may have the same reference numerals, and their descriptions may not be repeated in the description of FIG. 4B. A difference in the diagnostic device 401 relative to the diagnostic device 400 may be seen at the lyophilized reagent 414. In some embodiments, the reagent 414 may be held in the internal cavity 404 of the container 402, and a buffer fluid 480 in the first compartment 406 may be configured to activate the reagent 414. For example, after obtaining a sample from a patient (e.g., by swabbing a nasal cavity of the patient using the sample element 432 of the sample swab 430), the sample swab 430 may be inserted into the internal cavity 404 the container 402, as depicted by the dashed arrow in FIG. 48B, and the sealing device 460 may then be zipped shut. Upon rupturing of the first compartment 406, the buffer fluid 408 may be released into the internal cavity 404 to interact with the reagent 414 and the sample to form a sample fluid. Upon rupturing of the second compartment 408, a diluent fluid 482 in the second compartment 408 may be released to into the internal cavity 404 to interact with the sample fluid to form a diluted sample fluid. In FIG. 4B, the sealing device 460 is depicted in the zipped-open state, enabling the internal cavity 404 to be accessible for insertion of the sample swab 430.

According to some embodiments of the present technology, a rapid diagnostic test kit may include any one or any combination of: the diagnostic device 400 or the diagnostic device 401; the sample swab 430; the reagent 414, which may be provided in the internal cavity 404 or in a separate package to be added to the internal cavity 404; the heater 450; software for electronically reading the LFA strip 412; and instructions (in electronic form and/or in paper form) for using the test kit. Components of the test kit may be packaged individually or together.

2.3 Diagnostic Devices with Burstable Compartments and a Manifold

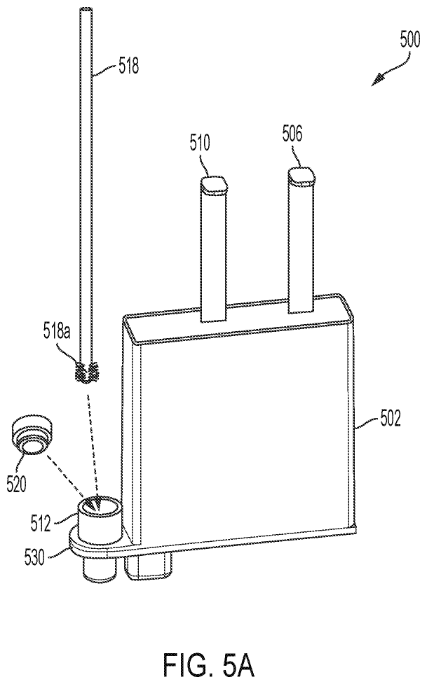

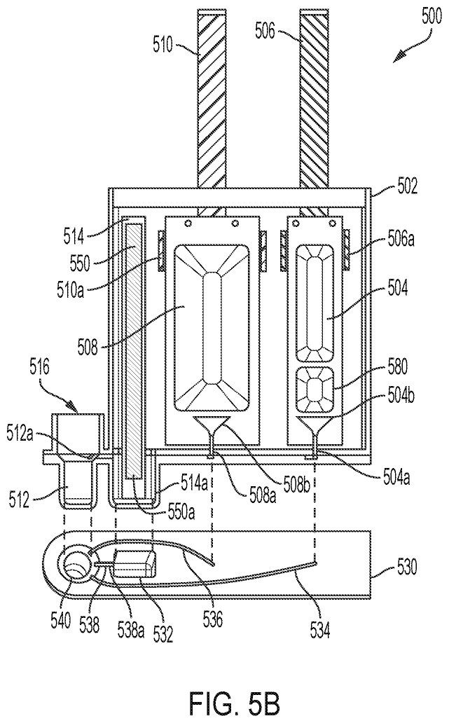

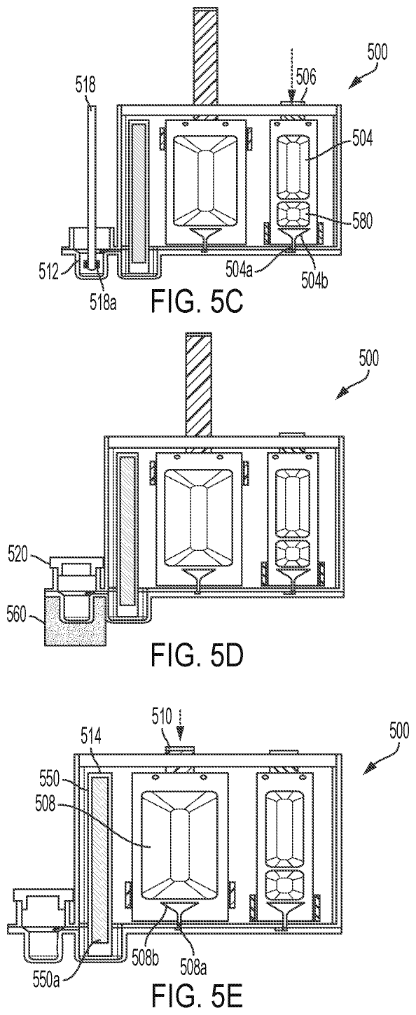

FIGS. 5A through 5E schematically depict a diagnostic device 500 that utilizes burstable compartments to hold fluids and other test materials for a diagnostic test procedure, according to some embodiments of the present technology. The diagnostic device 500 may comprise a housing 502 configured to support a burstable first compartment 504 containing a first fluid, a burstable second compartment 508 containing a second fluid, and a burstable test and readout compartment 514 supported by the housing 502 and containing a LFA strip 550. A sample compartment 512 may be supported by the housing 502.

In some embodiments of the present technology, a movable first force applicator 506 may be supported by the housing 502 and may be configured to move to apply a first bursting force to the first compartment 504. In some embodiments, the first force applicator 506 may have a rest position, depicted in FIG. 5B, at which no force or a minimal (e.g., non-bursting) force is applied to the first compartment 504. In some embodiments, movement of the first force applicator 506 from the rest position to a final position, depicted in FIG. 5C, may cause a portion of the first force applicator 506 to bear against the first compartment 504 to compress or squeeze the first compartment 504. When a force applied by the first force applicator 506 exceeds a first bursting force, the first compartment 504 may rupture and the first fluid may be released from the first compartment 504. In some embodiments, the first fluid may flow to an outlet 504a of the first compartment 504. In some embodiments, the first fluid may flow through a tapered conduit or funnel 504b, which may direct the first fluid to the outlet 504a.

Similarly, according to some embodiments of the present technology, a movable second force applicator 510 may be supported by the housing 502 and may be configured to move to apply a second bursting force to the second compartment 508. In some embodiments, the second force applicator 510 may have a rest position, depicted in FIG. 5B, at which no force or a minimal (e.g., non-bursting) force is applied to the second compartment 508. In some embodiments, movement of the second force applicator 510 from the rest position to a final position, depicted in FIG. 5E, may cause a portion of the second force applicator 510 to bear against the second compartment 508 to compress or squeeze the second compartment 508. When a force applied by the second force applicator 510 exceeds a second bursting force, the second compartment 508 may rupture and the second fluid may be released from the second compartment 508. In some embodiments, the second fluid may flow to an outlet 508a of the second compartment 508. In some embodiments, the second fluid may flow through a tapered conduit or funnel 508b, which may direct the second fluid to the outlet 508a.

According to some embodiments of the present technology, the first force applicator 506 and/or the second force applicator 510 may be a mechanical device configured to be moved directly by a user (e.g., by pushing or pressing a handle portion of the first force applicator 506 and/or the second force applicator 510) or may be an electromechanical device configured to be moved indirectly by a user (e.g., by activating a switch that causes an electronic actuator to move the first force applicator 506 and/or the second force applicator 510). In some embodiments, the first force applicator 506 may have a contact end 506a shaped to apply a force over an entire width of the first compartment 504 or over a portion of the entire width. In some embodiments, the second force applicator 510 may have a contact end 510a shaped to apply a force over an entire width of the second compartment or over a portion of the entire width.

According to some embodiments of the present technology, the sample compartment 512 may be comprised of a cavity 516 configured to receive a sample to be tested. In some embodiments, the cavity 516 may be configured to receive a swab element 518a of a sample swab 518 carrying the sample. In some embodiments, the cavity 516 may be sealed by a removable cover 520 during transit and/or storage of the apparatus 500. During the testing procedure, the cover 520 may be removed to enable the swab element to be inserted in the cavity 516. After receiving the sample, the cavity 516 may be resealed by the cover 520 to prevent contamination of the sample and to prevent loss of the sample from vaporization and/or spillage.

According to some embodiments of the present technology, the apparatus 500 may be comprised of a manifold 530 configured to mate with the housing 502. In some embodiments, the manifold 530 may be structured to mount to or receive a portion of the housing 502 such that, when the housing 502 is in a mounted position on the manifold 530, the manifold 530 may connect the sample compartment 512 to each of the first compartment 504, the second compartment 508, and the test and readout compartment 514. In some embodiments, a mounting procedure to place the housing 502 in the mounted position on the manifold 530 may cause a portion of the manifold 530 to exert a force on the test and readout compartment 514 to burst the test and readout compartment 514 at an intake end 514a of the test and readout compartment 514. For example, the intake end 514 may be comprised of a frangible seal that ruptures during insertion of the intake end 514 into a recess 532 of the manifold 530 during the mounting procedure. In some embodiments, the sample compartment 512 may sit in a recess 540 of the manifold 530 when the housing 502 is in the mounted position.

According to some embodiments of the present technology, the manifold 530 may be comprised of a first channel 534 configured to connect the outlet 504a of the first compartment 504 to the sample compartment 512, a second channel 536 configured to connect the outlet 508a of the second compartment 508 to the sample compartment 512, and a third channel 538 configured to connect an outlet 512a of the sample compartment 512 to the recess 532 in the manifold 530 in which the test and readout compartment 514 may sit when the housing 502 is in the mounted position.

According to some embodiments of the present technology, a lyophilized reagent (not shown) may be included in the cavity 516 of the sample compartment 512. The first fluid in the first compartment 514 may be a buffer fluid configured to activate the reagent. In some embodiments, when the first force applicator 506 moves to apply the first force to burst the first compartment 504, the buffer fluid may flow out of the first compartment 504 to the sample compartment 512 via the first channel 534 of the manifold 530. The reagent may interact with the buffer fluid in the sample compartment 512. In some embodiments, the reagent may be an amplification reagent. In some embodiments, the sample may interact with the buffer solution and the reagent to form a sample fluid.

According to some embodiments of the present technology, the housing 502 may support a burstable third compartment 580 containing a reactant. As depicted in FIG. 5B, the third compartment 580 may be in a movement path of the first force applicator 506 such that when the first force applicator 506 is moved from the rest position to the final position the first force applicator 506 may bear against the third compartment 580 and apply a third bursting force to rupture the third compartment 580. In some embodiments, the first fluid in the first compartment 504 may be a buffer fluid and, when the first force applicator 506 is moved to apply the first force to burst the first compartment 504 and the third force to burst the third compartment 580, the buffer fluid may flow out of the first compartment 504 and may contact and interact with the reagent to form a reagent fluid. The reagent fluid may flow to the sample compartment 512 via the first channel 534 of the manifold 530. The reagent fluid may interact with the sample in the sample compartment 512 to form a sample fluid.

According to some embodiments of the present technology, the second fluid may be a diluent fluid. When the second force applicator 510 is moved to apply the second force to burst the second compartment 508, the diluent fluid may flow out of the second compartment 508 to the sample compartment 512 via the second channel 536 of the manifold 530. The diluent fluid may interact with the sample fluid in the sample compartment 512 to form a diluted sample fluid.

According to some embodiments of the present technology, as the diluent fluid mixes with the sample fluid to form the diluted sample fluid, an amount of the diluted sample fluid in the sample compartment 512 may exceed a capacity or volume of the sample compartment 512, which may be a known or predetermined quantity based on a geometry of the sample compartment 512. In some embodiments, when the cavity 516 of the sample compartment 512 is sealed with the cover 520 and the amount of the diluted sample fluid exceeds the capacity of the sample compartment 512, a portion of the diluted sample fluid may flow out of the sample compartment 512 to the test and readout compartment 514 via the third channel 538 and the recess 532 of the manifold 530. In some embodiments, when the sample compartment 512 is sealed and the second force applicator 510 is moved to apply the second force to burst the second compartment 508, the second force applicator 510 may squeeze the diluent fluid out of the second compartment 508 and into the second channel 536 of the manifold 530. A differential pressure between the second channel 536 and the third channel 538 may cause the diluted sample fluid in the sample compartment 512 to flow out of the outlet 512a to the recess 532, and may enter the test and readout compartment 514 via, e.g., a burst hole formed at the frangible seal of the test and readout compartment 514. For example, the burst hole may face a surface of the recess 532 in which the test and readout compartment 514 sits. In some embodiments, an intake end 550a of the LFA strip 550 may be disposed proximate an outlet end 538a of the third channel 538, which may enable a fluid connection between the sample compartment 512 and the test and readout compartment 514 via the recess 532. The diluted sample fluid may enter the test and readout compartment 514 via the burst hole and may be absorbed at the intake end 550a of the LFA strip 550. The diluted sample fluid may be conveyed to test regions of the LFA strip 550 by capillary action.

According to some embodiments of the present technology, a portion of the sample compartment 512 may be configured to be received in a heater 560, as depicted in FIG. 5D. In some embodiments, an external surface of the recess 540 of the manifold 530 may be configured to be received in the heater 560, such that the sample compartment 512 may be heated by the heater 560 via material forming the recess 540. In some embodiments, the heater 560 may be comprised of an electronic interlock device configured to detect a presence of the cap 520 on the sample compartment 512 and to prevent the heater 560 from heating the sample compartment 512 when the cavity 516 of the sample compartment 512 is not sealed by the cap 520. The interlock device may prevent loss of the sample fluid through vaporization during heating of the sample compartment 512. In some embodiments, when the interlock device detects the presence of the cap 520 sealing the cavity 516, the heater 560 may be activated automatically to perform a heating procedure to heat the sample compartment 512.

According to some embodiments of the present technology, the test and readout compartment 514 may be comprised of a window (not shown) through which the LFA strip 550 may be visible, such that the LFA strip 550 may be readable by a human and/or an electronic reader through the window.

According to some embodiments of the present technology, a rapid diagnostic test kit may include any one or any combination of: the diagnostic device 500; the manifold 530; the sample swab 518; the heater 560; software for electronically reading the LFA strip 550; and instructions (in electronic form and/or in paper form) for using the test kit. Components of the test kit may be packaged individually or together.

2.4 Methods of Using a Diagnostic Device with Burstable Compartments

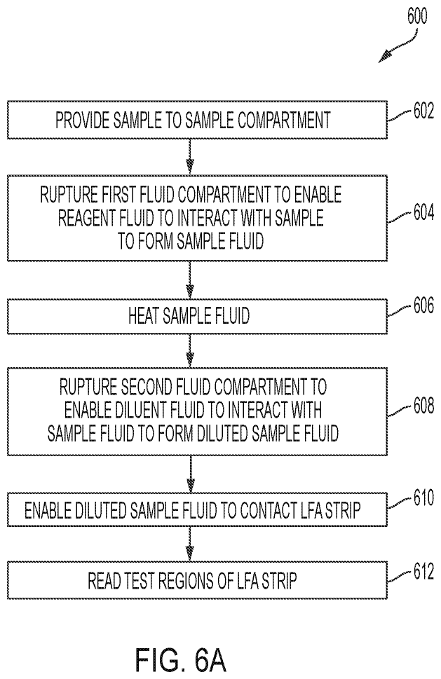

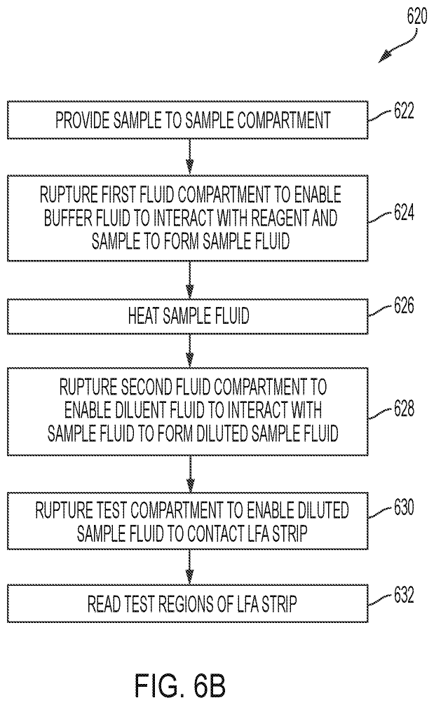

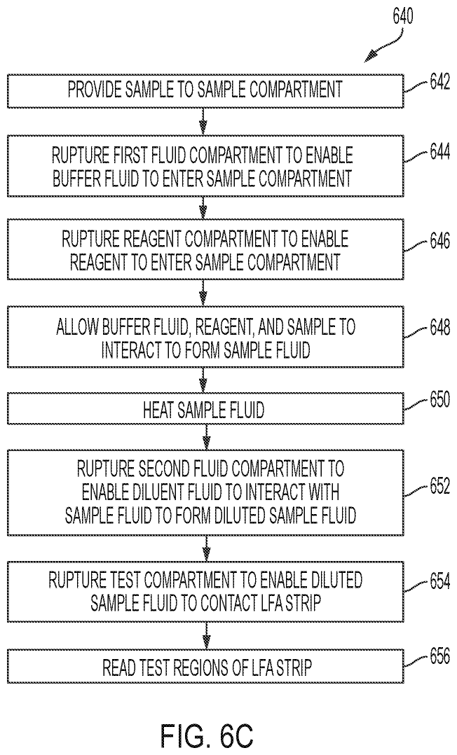

FIGS. 6A through 6C show flow diagrams summarizing example methods of using diagnostic devices with burstable compartments, according to some embodiments of the present technology. The methods may be performed by any one or any combination of: a subject to be tested, a nurse, a doctor, a teacher, a parent, a friend, etc. That is, no prior knowledge of medical technology or scientific methods is required. As will be appreciated, although the flow diagrams may show a particular order of steps or acts to be performed, the steps or acts need not be performed in the orders shown in the flow diagrams.

FIG. 6A shows a flow diagram summarizing a testing method 600, according to some embodiments of the present technology. In some embodiments, the method 600 may be performed using any of the diagnostic devices 100, 200, 300, 400, 401, 500. At act 602, a sample may be provided to a sample compartment of an apparatus. At act 604, a burstable first fluid compartment may be ruptured to enable a reagent fluid (e.g., an amplification fluid) in the first fluid compartment to be released to interact with the sample to form a sample fluid. In some embodiments, formation of the sample fluid may take place in the sample compartment. Optionally, at act 606, the sample fluid may be heated by a heater (e.g., to promote an amplification reaction). Also optionally, the sample compartment may be sealed (e.g., an opening may be zipped or covered) prior to heating of the sample (e.g., after the sample is provided to the sample compartment). At act 608, a burstable second fluid compartment may be ruptured to enable a diluent fluid in the second fluid compartment to be released to interact with the sample fluid to form a diluted sample fluid. In some embodiments, formation of the diluted sample fluid may take place in the sample compartment and/or in a conduit in fluid communication with the sample compartment. At act 610, the diluted sample fluid may be permitted to interact with a LFA strip. For example, a test compartment in which the LFA strip is housed may be ruptured to enable the diluted sample fluid to reach the LFA strip in the test compartment. In some embodiments, an intake end of the LFA strip may come into contact with the diluted sample fluid, and the diluted sample fluid may travel to test regions of the LFA strip by capillary action. At act 612, after interaction with the diluted sample fluid, the LFA strip may be read, as described herein. For example, the test regions of the LFA strip may be read by an electronic device (e.g., a smartphone camera), which may be programmed to recognize a particular appearance of a test region to indicate a presence of a pathogen in the sample. As will be appreciated, bursting of any one or any combination of the first fluid compartment, the second fluid compartment, and the test compartment may take place at respective burstable seals of the compartments or may take place anywhere on the compartments. As discussed above, bursting of any one or any combination of the first fluid compartment, the second fluid compartment, and the test compartment may occur upon application of a rupturing force (e.g., any one or any combination of: a squeezing force, a pinching force, a rubbing force, a bending force, etc.). The rupturing force may be applied directly by a user or indirectly by a device (e.g., a squeegee, a plunger, etc.) operated by a user. In some cases, the rupturing force may be electronically actuated (e.g., electronic plunger, electronic pincher, etc.) under control of a user.

FIG. 6B shows a flow diagram summarizing a testing method 620, according to some embodiments of the present technology. In some embodiments, the method 620 may be performed using any of the diagnostic devices 100, 200, 300, 400, 401, 500. At act 622, a sample may be provided to a sample compartment of a diagnostic device. At act 624, a burstable first fluid compartment may be ruptured to enable a buffer fluid in the first fluid compartment to be released to interact with a lyophilized amplification reagent to activate the reagent. For example, the reagent may be present in the sample chamber, and rupturing of the first fluid compartment may release the buffer fluid into the sample chamber. A sample fluid may be formed from interaction of the reagent, the buffer fluid, and the sample. Optionally, at act 626, the sample fluid may be heated by a heater (e.g., to promote an amplification reaction). Also optionally, the sample compartment may be sealed (e.g., an opening may be zipped or covered) prior to heating of the sample (e.g., after the sample is provided to the sample compartment). At act 628, a burstable second fluid compartment may be ruptured to enable a diluent fluid in the second fluid compartment to be released to interact with the sample fluid to form a diluted sample fluid. In some instances, formation of the diluted sample fluid may take place in the sample compartment and/or in a conduit in fluid communication with the sample compartment. At act 630, the diluted sample fluid may be permitted to interact with a LFA strip. For example, a test compartment in which the LFA strip is housed may be ruptured to enable the diluted sample fluid to reach the LFA strip in the test compartment. In some embodiments, an intake end of the LFA strip may come into contact with the diluted sample fluid, and capillary action may cause the diluted sample fluid to travel to test regions of the LFA strip. At act 632, after interaction with the diluted sample fluid, the LFA strip may be read, as described herein. For example, the test regions of the LFA strip may be read by an electronic device (e.g., a smartphone camera), which may be programmed to recognize a particular appearance of a test region to indicate a presence of a pathogen in the sample. As will be appreciated, bursting of any one or any combination of the first fluid compartment, the second fluid compartment, and the test compartment may take place at respective burstable seals of the compartments or may take place anywhere on the compartments. As discussed above, bursting of any one or any combination of the first fluid compartment, the second fluid compartment, and the test compartment may occur upon application of a rupturing force (e.g., any one or any combination of: a squeezing force, a pinching force, a rubbing force, a bending force, etc.). The rupturing force may be applied directly by a user or indirectly by a device (e.g., a squeegee, a plunger, etc.) operated by a user. In some cases, the rupturing force may be electronically actuated (e.g., electronic plunger, electronic pincher, etc.) under control of a user.

FIG. 6C shows a flow diagram summarizing a testing method 640, according to some embodiments of the present technology. In some embodiments, the method 640 may be performed using any of the diagnostic devices 100, 200, 300, 400, 401, 500. At act 642, a sample may be provided to a sample compartment of an apparatus. At act 644, a burstable first fluid compartment may be ruptured to enable a buffer fluid in the first fluid compartment to be released. At act 646, a burstable reagent compartment may be ruptured to enable a lyophilized reagent to be released. The buffer fluid may be configured to interact with the reagent to activate the reagent in the sample chamber. At act 648, a sample fluid may be formed from interaction of the reagent, the buffer fluid, and the sample. Optionally, at act 650, the sample fluid may be heated by a heater. Also optionally, the sample compartment may be sealed (e.g., an opening may be zipped or covered) prior to heating of the sample (e.g., after the sample is provided to the sample compartment). At act 652, a burstable second fluid compartment may be ruptured to enable a diluent fluid in the second fluid compartment to be released to interact with the sample fluid to form a diluted sample fluid. In some instances, formation of the diluted sample fluid may take place in the sample compartment and/or in a conduit in fluid communication with the sample compartment. At act 654, the diluted sample fluid may be permitted to interact with a LFA strip. For example, a test compartment in which the LFA strip is housed may be ruptured to enable the diluted sample fluid to reach the LFA strip in the test compartment. In some embodiments, an intake end of the LFA strip may come into contact with the diluted sample fluid, and capillary action may cause the diluted sample fluid to travel to test regions of the LFA strip. At act 656, after interaction with the diluted sample fluid, the LFA strip may be read, as described herein. For example, the test regions of the LFA strip may be read by an electronic device (e.g., a smartphone camera), which may be programmed to recognize a particular appearance of a test region to indicate a presence of a pathogen in the sample. As will be appreciated, bursting of any one or any combination of the first fluid compartment, the second fluid compartment, the reagent compartment, and the test compartment may take place at respective burstable seals of the compartments or may take place anywhere on the compartments. As discussed above, bursting of any one or any combination of the first fluid compartment, the second fluid compartment, the reagent compartment, and the test compartment may occur upon application of a rupturing force (e.g., any one or any combination of: a squeezing force, a pinching force, a rubbing force, a bending force, etc.). The rupturing force may be applied directly by a user or indirectly by a device (e.g., a squeegee, a plunger, etc.) operated by a user. In some cases, the rupturing force may be electronically actuated (e.g., electronic plunger, electronic pincher, etc.) under control of a user.

2.5 Method of Manufacturing a Diagnostic Device with Burstable Compartments

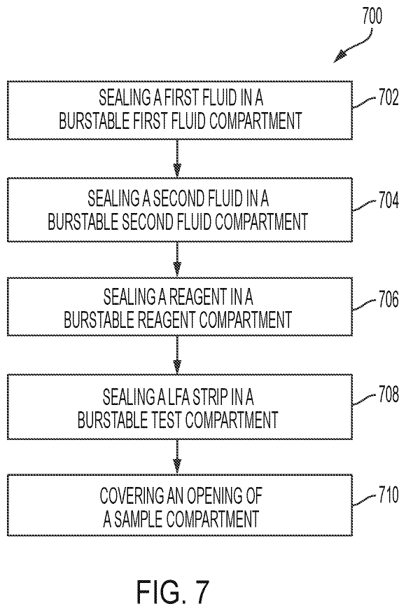

FIG. 7 shows a flow diagram summarizing an example method 700 of manufacturing portions of a diagnostic device with burstable compartments (e.g., any of the apparatuses 100, 200, 300, 400, 401, 500), according to some embodiments of the present technology. As will be appreciated, although the flow diagram may show a particular order of steps or acts to be performed, the steps or acts need not be performed in the order shown. At act 702, a first fluid may be sealed in a burstable first fluid compartment (e.g., 104, 306, 406, 504) of the diagnostic device. For example, the first fluid compartment may be part of a container (e.g., the container 302, 402) or part of a housing (e.g., 502) or attached to a sample chamber (e.g., 102). In some embodiments, the first fluid may be a buffer fluid (e.g., a fluid configured to activate a lyophilized reagent). In some embodiments, the first fluid may be a reagent fluid (e.g., an amplification fluid). In some embodiments, sealing of the first fluid in the first fluid compartment may comprise providing the first fluid compartment with a burstable seal. At act 704, a second fluid may be sealed in a burstable second compartment (e.g., 110, 308, 408, 508) of the apparatus. For example, as with the first fluid compartment, the second fluid compartment may be part of a container (e.g., the container 302, 402) or part of a housing (e.g., 502) or attached to a sample chamber (e.g., 102). In some embodiments, the second fluid may be a diluent fluid. In some embodiments, sealing of the second fluid in the second fluid compartment may comprise providing the second fluid compartment with a burstable seal. Optionally, at act 706, a lyophilized reagent may be sealed in a burstable reagent compartment (e.g., 316, 416, 580) of the diagnostic device. For example, as with the first and second fluid compartments, the reagent compartment may be part of a container (e.g., the container 302, 402) or part of a housing (e.g., 502). In some embodiments, the reagent may be an amplification reagent. In some embodiments, sealing of the reagent in the reagent compartment may comprise providing the reagent compartment with a burstable seal. In another option, not shown in FIG. 7, a lyophilized reagent may be added to a container (e.g., the sample chamber 102) without being sealed in a burstable compartment. At act 708, a LFA strip may be sealed in a burstable test compartment (e.g., 116, 310, 410, 514) of the diagnostic device. For example, as with the first and second fluid compartments, the test compartment may be part of a container (e.g., the container 302, 402) or part of a housing (e.g., 502) or attached to a sample chamber (e.g., 102). In some embodiments, sealing of the LFA strip in the test compartment may comprise providing the test compartment with a burstable seal. Optionally, at act 710, a sample compartment (e.g., 102, 304, 404, 512) of the diagnostic device may be covered (e.g., with a removable cap or a zipper-type seal or the like) to prevent contamination of an internal cavity of the sample compartment until the apparatus is to be used in a test procedure.

### 3. Test Methodologies

The diagnostic devices described herein may be used to detect whether a test subject is afflicted with a communicable disease by detecting whether a target nucleic-acid sequence corresponding to a pathogen of interest and indicative of the disease is present in a sample obtained from the test subject. The sample may be comprised of, for example, saliva and/or mucus obtained from the test subject, and/or may be cells obtained from the test subject by other means (e.g., by scraping the test subject's skin). Target nucleic-acid sequences and techniques that may be used for their detection are described below.

Target nucleic-acid sequences may be associated with a variety of diseases or disorders. In some embodiments of the present technology, the diagnostic devices described herein may be used to diagnose at least one disease or disorder caused by a pathogen. In some embodiments, the diagnostic devices may be configured to detect a nucleic acid encoding a protein (e.g., a nucleocapsid protein) of SARS-CoV-2, which is the virus that causes COVID-19. In some embodiments, the diagnostic devices may be configured to identify particular strains of a pathogen (e.g., a virus). In some embodiments, a diagnostic device may utilize and be comprised of an assay vehicle (e.g., an LFA strip) comprised of a first test line configured to detect a nucleic-acid sequence of SARS-CoV-2 and a second test line configured to detect a nucleic-acid sequence of a SARS-CoV-2 virus having a D614G mutation (i.e., a mutation of the 614^{th} amino acid from aspartic acid (D) to glycine (G)) in its spike protein. In some embodiments, one or more target nucleic-acid sequences may be associated with a single-nucleotide polymorphism (SNP). In certain cases, the diagnostic devices may be used for rapid genotyping to detect whether a SNP, which may affect medical treatment, is present.

According to some embodiments of the present technology, the diagnostic devices described herein may be configured to diagnose two or more diseases or disorders. This may be referred to herein as multiplexed testing. In certain cases, for example, a diagnostic device may utilize and be comprised of an LFA strip comprised of a first test line configured to detect a nucleic-acid sequence of SARS-CoV-2, a second test line configured to detect a nucleic-acid sequence of an influenza virus (e.g., an influenza A virus), and a third line configured to detect a nucleic-acid sequence of another influenza virus (e.g., an influenza B virus) or a nucleic acid sequence of a bacterium.

3.1 Lysis of Samples

According to some embodiments of the present technology, lysis may be performed on a sample by chemical lysis techniques (e.g., exposing the sample to one or more lysis reagents) and/or thermal lysis techniques (e.g., heating the sample). In chemical lysis, lysis may be performed by one or more lysis reagents, discussed below.

According to some embodiments of the present technology, a lysis reagent may be in solid form (e.g., lyophilized, dried, crystallized, air jetted, etc.). For example, a solid lysis reagent may be in the form of a pellet, or capsule, or gelcap, or tablet. In some embodiments, a solid lysis reagent may be included in a caged cap, as described above. In some embodiments, a lysis reagent may be comprised of one or more additional reagents (e.g., a reagent to reduce or eliminate cross contamination).

According to some embodiments of the present technology, a solid lysis reagent may be shelf stable for a relatively long period of time. In some embodiments, a lysis pellet, or capsule, or gelcap, or tablet may be shelf stable for at least 1 month, at least 3 months, at least 6 months, at least 1 year, at least 5 years, or at least 10 years. In some embodiments, a solid lysis reagent may be thermostabilized and may be stable across a wide range of temperatures. In some embodiments, a lysis pellet, or capsule, or gelcap, or tablet may be stable at a temperature of at least 0° C., at least 10° C., at least 20° C., at least 37° C., at least 65° C., or at least 100° C. As will be appreciated, a solid lysis reagent may be activated before or during use with a sample by contact with a buffer fluid.

As noted above, thermal lysis may be accomplished by applying heat to a sample. According to some embodiments of the present technology, thermal lysis may be performed by applying a lysis heating protocol comprised of heating the sample at one or more temperatures for one or more time periods or durations using any suitable heater (e.g., the heater 450).

3.2 Nucleic-Acid Amplification

Following lysis, one or more target nucleic acids (e.g., a nucleic acid of a target pathogen) may be amplified, according to some embodiments of the present technology. In some embodiments, DNA may be amplified according to any nucleic-acid amplification method known in the art. For example, nucleic-acid amplification methods that may be employed may include isothermal amplification methods, which include: loop-mediated isothermal amplification (LAMP), recombinase polymerase amplification (RPA), nicking enzyme amplification reaction (NEAR), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), whole genome amplification (WGA), and CRISPR-related amplification, such as CRISPR-Cas9-triggered nicking endonuclease-mediated strand displacement amplification (CRISDA). In some embodiments, an isothermal amplification method that may be performed in a test procedure may be comprised of applying heat to a sample. For example, heat may be applied to a sample fluid containing the sample. In some embodiments, the isothermal amplification method may be comprised of applying an amplification heating protocol, which may be comprised of heating the sample at one or more temperatures for one or more time periods using any appropriate heater (e.g., the heater 450).

In embodiments where a target pathogen may have RNA as its genetic material, the target pathogen's RNA may need to be reverse transcribed to DNA prior to amplification.

3.3 Molecular Switches

As described herein, a sample may undergo lysis and amplification prior to detection of a target nucleic-acid sequence. Reagents associated with lysis and/or amplification may be in solid form (e.g., lyophilized, dried, crystallized, air jetted, etc.). According to some embodiments of the present technology, one or more (and, in some cases, all) of the reagents necessary for lysis and/or amplification may be present in a single pellet, capsule, gelcap, or tablet. In some embodiments, the pellet, capsule, gelcap, or tablet may be comprised of two or more enzymes, and it may be necessary for the enzymes to be activated in a particular order. Therefore, in some embodiments, the enzyme-containing tablet, pellet, capsule, or gelcap may further be comprised of one or more molecular switches.

Molecular switches, as used or described herein, may be molecules that, in response to certain conditions, reversibly switch between two or more stable states. According to some embodiments of the present technology, a condition that causes a molecular switch to change its configuration may be associated with any one or any combination of: pH, light, temperature, an electric current, microenvironment, and presence of ions and/or other ligands. In some embodiments, the condition may be heat. In some embodiments, the molecular switches may be comprised of aptamers. Aptamers may refer generally to oligonucleotides or peptides that may bind to specific target molecules (e.g., the enzymes described herein). The aptamers, upon exposure to heat or other conditions, may dissociate from the enzymes. With use of molecular switches, one or more of the processes described herein (e.g., lysis, decontamination, reverse transcription, amplification, etc.) may be performed in a single test tube with a single enzymatic tablet, pellet, capsule, or gelcap.

3.4 CRISPR/Cas Techniques

According to some embodiments of the present technology, CRISPR/Cas detection techniques may be used to detect a target nucleic-acid sequence. For example, one or more CRISPR/Cas detection reagents may be included on an LFA strip. CRISPR generally may refer to Clustered Regularly Interspaced Short Palindromic Repeats, and Cas generally may refer to a particular family of proteins. In some embodiments, a CRISPR/Cas detection platform or technique may be combined with an isothermal amplification method to create a single-step reaction (Joung et al., “Point-of-care testing for COVID-19 using SHERLOCK diagnostics,” 2020). For example, amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

### 4. Reagents

According to some embodiments of the present technology, the diagnostic devices described herein may comprise and/or utilize reagents (e.g., lysis reagents, nucleic-acid amplification reagents, CRISPR/Cas detection reagents, and the like) in various test procedures of a diagnostic test. In some embodiments, one or more of the reagents may be contained within a diagnostic device (e.g., in a reaction vial of the diagnostic device). In some embodiments, one or more of the reagents may be provided separately (e.g., in one or more caged caps, in one or more separate vials, etc.). For example, a diagnostic device may be comprised of one or more caged caps comprising one or more lysing reagents and/or one or more amplification reagents.

According to some embodiments of the present technology, at least one (and, in some instances, each) of the reagents used in a diagnostic test may be in liquid form (e.g., in solution). In some embodiments, at least one (and, in some instances, each) of the reagents used in a diagnostic test may be in solid form (e.g., lyophilized, dried, crystallized, air jetted, and the like) and may be activated with buffer fluids prior to or during use.

4.1 Lysing Reagents

According to some embodiments of the present technology, the reagents may be comprised of one or more lysis reagents. A lysis reagent may refer generally to a reagent that promotes cell lysis either alone or in combination with one or more other reagents and/or one or more conditions (e.g., heating). In some embodiments, the lysis reagents may be comprised of one or more enzymes. Non-limiting examples of suitable enzymes may include lysozyme, lysostaphin, zymolase, cellulose, protease, and glycanase. In some embodiments, the lysis reagent(s) may be comprised of one or more detergents. Non-limiting examples of suitable detergents may include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the lysis reagents may be comprised of an RNase inhibitor (e.g., a murine RNase inhibitor). In some embodiments, a concentration of the RNase inhibitor may be is at least 0.1 U/μL, at least 1.0 U/μL, or at least 2.0 U/μL. In some embodiments, the concentration of the RNase inhibitor may be in a range from 0.1 U/μL to 0.5 U/μL, 0.1 U/μL to 1.5 U/μL, or 1.0 U/μL to 2.0 U/μL. In some embodiments, the lysis reagents may comprise Tween (e.g., Tween 20, Tween 80).

4.2 Contamination-Prevention Reagents

According to some embodiments of the present technology, the reagents may be comprised of at least one reagent that works to reduce or eliminate potential carryover contamination from prior tests (e.g., prior tests conducted with a common apparatus and/or in a same area). In some embodiments, the reagents may be comprised of thermolabile uracil DNA glycosylase (UDG). In some embodiments, UDG may prevent carryover contamination from prior tests by degrading products that have already been amplified (i.e., amplicons) while leaving unamplified samples untouched and ready for amplification. In some embodiments, a concentration of UDG may be at least 0.01 U/μL, at least 0.03 U/μL, or at least 0.05 U/μL. In some embodiments, the concentration of UDG may be in a range from 0.01 U/μL to 0.02 U/μL or 0.01 U/μL to 0.04 U/μL.

4.3 Reverse Transcription Reagents

According to some embodiments of the present technology, the reagents may be comprised of one or more reverse transcription reagents. As noted above, a target pathogen may have RNA as its genetic material, which may need to be reverse transcribed to DNA prior to amplification. In some embodiments, the reverse transcription reagents may facilitate such reverse transcription. In some embodiments, the reverse transcription reagents may be comprised of a reverse transcriptase, a DNA-dependent polymerase, and/or a ribonuclease (RNase). A reverse transcriptase may refer generally to an enzyme that transcribes RNA to complementary DNA (cDNA) by polymerizing deoxyribonucleotide triphosphates (dNTPs). An RNase may refer generally to an enzyme that catalyzes the degradation of RNA. In some embodiments, an RNase may be used to digest RNA from an RNA-DNA hybrid.

4.4 Nucleic-Acid Amplification Reagents

According to some embodiments of the present technology, the reagents may comprise one or more nucleic-acid amplification reagents. In some embodiments, the nucleic-acid amplification reagents may comprise LAMP reagents, RPA reagents, and NEAR reagents, known in the art. In some embodiments, an enzyme (e.g., Bsm DNA polymerase) may serve as an amplification reagent.

4.5 Reagent Stability Enhancers

According to some embodiments of the present technology, the reagents may comprise one or more additives that may enhance reagent stability (e.g., protein stability). Non-limiting examples of suitable additives may include trehalose, polyethylene glycol (PEG), polyvinyl alcohol (PVA), and glycerol.

4.6 Buffers

According to some embodiments of the present technology, the reagents may comprise one or more reaction buffers. Non-limiting examples of suitable buffers may include phosphate-buffered saline (PBS) and Tris. In some embodiments, the buffers may be buffer fluids. In some embodiments, the buffers may have a relatively neutral pH. In some embodiments, the buffers may have a pH in a range from 5.0 to 7.0, 6.0 to 8.0, 7.0 to 9.0, or 8.0 to 9.0. In some embodiments, the buffers may comprise one or more salts. Non-limiting examples of suitable salts may include magnesium acetate tetrahydrate, potassium acetate, and potassium chloride. In some embodiments, the buffers may comprise Tween (e.g., Tween 20, Tween 80). In some embodiments, the buffers may comprise an RNase inhibitor. In some embodiments, Tween and/or an RNase inhibitor may facilitate cell lysis. In a particular, non-limiting embodiment of the present technology, the buffers may comprise 25 mM Tris buffer, 5% (w/v) poly(ethylene glycol) 35,000 kDa, 14 mM magnesium acetate tetrahydrate, 100 mM potassium acetate, and greater than 85% volume nuclease free water.

### 5. Detection Devices

As noted above, according to some embodiments of the present technology, LFA strips may be used as assay vehicles to test for whether a target nucleic-acid sequence, corresponding to a pathogen of interest, is present in a sample obtained from a user. In some embodiments, the target nucleic acid-acid sequence may be amplified (i.e., amplicons) prior to detection via an LFA strip. In some embodiments, an LFA strip may provide results that may be read or interpreted in a non-clinical setting by a lay person (e.g., a person not trained in laboratory procedures). LFA strips may be comprised of reagents or substances for indicating the presence (or absence) of a target nucleic-acid sequence. In some embodiments, an LFA strip may be configured to detect two or more different target nucleic-acid sequences.

According to some embodiments of the present technology, an LFA strip useable with the diagnostic devices described herein may be comprised of one or more fluid-transporting layers, which may be comprised of one or more absorbent materials that allow a fluidic sample to move from one end of the LFA strip (e.g., an intake end) to an opposite end of the LFA strip. In some embodiments, fluid movement may be via wicking or capillary action. Non-limiting examples of suitable materials may include polyethersulfone, cellulose, polycarbonate, nitrocellulose, sintered polyethylene, and glass fibers.

According to some embodiments of the present technology, an LFA strip may be comprised of a plurality of sub-regions. In some embodiments, the fluidic sample may be introduced to a first sub-region (e.g., a region in contact with a sample pad) and may subsequently flow through a second sub-region (e.g., a particle conjugate pad) comprised of a plurality of labeled particles. In some embodiments, the particles may be comprised of gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some embodiments, as an amplicon-containing fluidic sample flows through the second sub-region, a labeled nanoparticle may bind to a label of an amplicon, thereby forming a particle-amplicon conjugate. In some embodiments, the fluidic sample may subsequently flow through a third sub-region comprised of one or more test lines. In some embodiments, a first test line may be comprised of a capture reagent (e.g., an immobilized antibody) configured to detect a first target nucleic-acid sequence. In some embodiments, a particle-amplicon conjugate may be captured by one or more capture reagents (e.g., immobilized antibodies), and an opaque marking may appear on the first test line. In some embodiments, the LFA strip may comprise one or more additional test lines configured to detect one or more different target nucleic-acid sequences. In some embodiments, the third sub-region of the LFA strip may further comprise one or more control lines. For example, a control line may be a human (or animal) nucleic-acid control line configured to detect a nucleic acid (e.g., RNase P) that is generally present in all humans (or animals). The control line may be used to confirm whether a human (or animal) sample was successfully collected, nucleic-acid sequences from the sample were amplified, and the amplicons were transported through the LFA strip successfully.

According to some embodiments of the present technology, a diagnostic device may be comprised of two or more LFA strips arranged in parallel, such that a sample fluid may flow in each LFA strip independently of the other LFA strip(s).

### 6. Test Kits

According to some embodiments of the present technology, the diagnostic devices described herein may be part of a test kit useable by a lay person, i.e., a person who is not trained in medical and/or laboratory techniques or procedures. The test kit may be a stand-alone test kit that does not require the use of additional laboratory equipment to perform a diagnostic test. In some embodiments, the test kit may be comprised of a swab device and a diagnostic device. One or more reagents necessary for the diagnostic test may be provided in the diagnostic device itself or may be provided in a reagent carrier (e.g., a caged cap) to be added by a user during a test procedure.

6.1 Heater

According to some embodiments of the present technology, a heater may be provided as part of a diagnostic device, e.g., to heat a sample solution (e.g., for lysis and/or amplification). In some embodiments, the heater may be a printed circuit board (PCB) heater. For example, the PCB heater may be comprised of a bonded PCB with a microcontroller, thermistors, and/or resistive heating elements. In some embodiments, the heater may be pre-programmed with one or more heating protocols. For example, the heater may be pre-programmed with a lysis heating protocol and/or an amplification heating protocol. The lysis heating protocol may be a set of one or more temperatures and one or more time periods that facilitate lysis of a sample. The amplification heating protocol may be a set of one or more temperatures and one or more time periods that facilitate amplification of a nucleic-acid sequence. In some embodiments, the heater may be comprised of an auto-start mechanism that performs heating according to a pre-programmed temperature profile needed for lysis and/or amplification upon activation of the auto-start mechanism by a user.

6.2 Instructions & Software

According to some embodiments of the present technology, a test kit may be comprised instructions associated with sample collection and/or operation of a diagnostic device. For example, the instructions may be comprised of directions for handling a swab device to obtain a sample from a subject as well as directions for providing a collected sample to a diagnostic device (or a component thereof) for further processing. The instructions may be provided in any form readable by a user. For example, the instructions may be written or published, verbal, audible (e.g., telephonic), digital, optical, visual (e.g., videotape, DVD, etc.), and/or provided via electronic communications (including Internet or web-based communications). In some embodiments, the instructions may combine graphical information with textual information. In some embodiments, the instructions may be provided as part of a software-based application.

According to some embodiments of the present technology, the instructions may be provided as part of a software-based application that may be downloaded to a smartphone or other type of portable electronic device, and contents of the downloaded application may guide a user through steps to use a diagnostic device and/or to perform test procedures of a diagnostic test. In some embodiments, the instructions may instruct a user when to add certain reagents and how to do so.

According to some embodiments of the present technology, a software-based application may be connected (e.g., via a wired or wireless connection) a diagnostic device to control the diagnostic device or components thereof and/or to read and analyze test results. In some embodiments, the application may be configured to process an image of an LFA strip captured by an imaging device (e.g., a smartphone camera, etc.) and to evaluate the image to provide a positive or negative test result for each of one or more test lines on the LFA strip.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A rapid diagnostic test apparatus, comprising:
a sample chamber configured with an opening through which a sample is received in the sample chamber;
a first fluid chamber containing a first fluid; and
a test and readout chamber containing a lateral-flow assay (LFA) strip,
wherein at least one of the first fluid chamber and the test and readout chamber is burstable and is configured to be in fluid connection with the sample chamber upon bursting.

2. The apparatus of claim 1, wherein:
the first fluid chamber is a flexible first fluid chamber and is configured to burst at a burstable first seal,
the first seal separates the sample chamber from the first fluid chamber, and
the first seal is configured break when a first bursting force is applied to the first fluid chamber.

3. The apparatus of claim 1, further comprising:
a burstable second fluid chamber containing a second fluid and configured to be in fluid connection with the sample chamber upon bursting.

4. The apparatus of claim 3, wherein:
the second fluid chamber is a flexible second fluid chamber and is configured to burst at a burstable second seal,
the second seal separates the sample chamber from the second fluid chamber, and
the second seal is configured break when a second bursting force is applied to the second fluid chamber.

5. The apparatus of claim 4, wherein:
the test and readout chamber is a flexible chamber and is configured to burst at a burstable third seal,
the third seal separates the sample chamber from the test and readout chamber, and
the third seal is configured to burst when the second bursting force is applied to the second fluid chamber.

6. The apparatus of claim 1, wherein:
the test and readout chamber is a flexible chamber and is configured to burst at a burstable third seal,
the third seal separates the sample chamber from the test and readout chamber, and
the third seal is configured to burst when a third bursting force is applied to the test and readout chamber.

7. The apparatus of claim 1, further comprising:
a conduit connecting the sample chamber and the test and readout chamber,
wherein an intake end of the LFA strip is disposed at an outlet end of the conduit.

8. The apparatus of claim 1, wherein the test and readout chamber is comprised of a window that enables a test region of the LFA strip in the test and readout chamber to be visible through the window.

9. The apparatus of claim 1, further comprising:
a sample swab comprised of a cap end and a sample end,
wherein:
the cap end of the sample swab is configured to seal the opening of the sample chamber, and
the sample end of the sample swab is configured to extend into a base portion of the sample chamber to deliver the sample into the base portion of the sample chamber.

10. The apparatus of claim 1, wherein:
the first chamber is configured to burst at a base end of the first chamber, and
the sample chamber is configured to have an upright position such that, upon bursting, gravity causes the first fluid to flow outward from the base end of the first chamber into the sample chamber.

11. The apparatus of claim 1, further comprising a heater configured to heat the sample chamber.

12. A rapid diagnostic test apparatus comprising:
a container configured to receive a sample in an internal cavity, the container being comprised of:
a rupturable first compartment holding a first fluid and configured to be in fluid communication with the internal cavity upon rupturing; and
a lateral-flow assay (LFA) strip disposed in a portion of the container.

13. The apparatus of claim 12, wherein:
the container is a resealable container configured to have an opened position in which the internal cavity of the container is accessible to receive the sample and a closed position in which the internal cavity is not accessible, and
the container is further comprised of:
a rupturable second compartment holding a second fluid and configured to be in fluid communication with the internal cavity upon rupturing, and
a rupturable third compartment holding the LFA strip and configured to be in fluid communication with the internal cavity upon rupturing.

14. The apparatus of claim 13, wherein:
at least one of the first, second, and third compartments is comprised of a burstable seal configured to rupture upon application of a rupturing force, and
the rupturing force is comprised of any one or any combination of:
a squeezing force,
a pinching force,
a jabbing force,
a rubbing force, and
a bending force.

15. The apparatus of claim 13, wherein:
the first compartment is configured to rupture into the internal cavity such that the first fluid flows into the internal cavity, and
the second compartment is configured to rupture into the internal cavity such that the second fluid flows into the internal cavity.

16. The apparatus of claim 12, further comprising:
a first force applicator movably attached to the container and configured to apply a first bursting force to the first compartment.

17. The apparatus of claim 12, further comprising:
a manifold configured to receive a base portion of the container such that, when the container is in a mounted position on the manifold, the manifold enables the first fluid to flow to the internal cavity from the first compartment.

18. A rapid diagnostic test kit, comprising:
a diagnostic test apparatus comprised of: a fluid compartment containing a fluid for a test procedure, and a test compartment containing a lateral-flow assay (LFA) strip, wherein at least one of the fluid compartment and the test compartment is burstable; and
a sample swab configured to collect a sample for the test procedure.

19. The test kit of claim 18, further comprising:
a heater configured to heat at least a apportion of the diagnostic test apparatus.

20. The test kit of claim 18, further comprising:
a reagent to be used in the test procedure.
